(19) United States
(12) Patent Application Publication
(10) Pub. No.: US 2024/0300187 A1
(43) Pub. Date: Sep. 12, 2024

(54) METHOD FOR PRODUCING A GATHERED MATERIAL, AND GATHERED MATERIAL

(71) Applicant: HERRMANN ULTRASCHALLTECHNIK GMBH & CO. KG, Karlsbad (DE)

(72) Inventors: Raquel ROUSSEL-GARCIA, Pforzheim (DE); Robin Alexander BODE, Pforzheim (DE)

(21) Appl. No.: 18/578,346

(22) PCT Filed: Mar. 10, 2022
(86) PCT No.: PCT/EP2022/056234

§ 371 Date: Jan. 11, 2024

(30) Foreign Application Priority Data
Jul. 13, 2021 (DE) 10 2021 118 030.9
Jul. 13, 2021 (DE) 10 2021 118 046.5

(51) Int. Cl. B29C 65/08; B29C 65/00

(52) U.S. Cl. CPC B29C 65/087; B29C 66/006; B29C 66/1122; B29C 66/344; B29C 66/433

(57) ABSTRACT

A method for producing a gatherable material comprising the steps of: a) providing two material web sections and a thread in between, b) providing processing device having a tool with a sealing surface and a counter-tool with a counter-sealing surface, the tool and the counter-tool being arranged relative to one another such that a gap is formed in between, the tool and/or the counter-tool having a groove for guiding the thread, c) passing the web sections and the thread through the gap, so that the two material web sections are welded together at two connecting surfaces, the thread being arranged between the two connecting surfaces such that there is a positive connection between the thread and the material web sections, a cross-section of the thread relative to a cross-section of the groove being selected such that an outer section of the thread is also welded to the material web sections.

### FIELD OF THE INVENTION

The present invention relates to a method for producing a gathered or gatherable material. Furthermore, the present invention relates to a corresponding gathered material.

The gathered or gatherable material consists of at least two material web sections and at least one thread, which is positioned between the material web sections. According to the method, the two material web sections are connected to each other at at least two connecting surfaces. The thread is arranged between the material web sections and between the two connecting surfaces, so that the thread is fixed between the material web sections in such a way that there is a positive connection between the thread and the material web sections in a first and a second direction, which is aligned perpendicular to the first direction.

The two material web sections can be realized by two material webs that are connected via the connecting surfaces. However, it is also possible to fold a material web in such a way that a first material web section of the material web lies opposite a second material web section of the same material web and the thread is arranged between the first and second material web sections.

To carry out the method, a tool with a sealing surface and a counter-tool with a counter-sealing surface are provided, the tool and the counter-tool being arranged in a processing position relative to one another in such a way that, to produce the gathered or gatherable material, the material web sections and the thread are fed through a gap formed by the sealing surface and the counter-sealing surface in a feed direction.

Furthermore, the tool and/or the counter tool has at least one groove for at least partially receiving the at least one thread, the groove being oriented in the feed direction and dividing the sealing surface and the counter-sealing surface, respectively, into partial sealing surfaces. In addition, the groove has a groove base and two side walls adjoining the groove base, wherein the side walls connect the groove base to the partial sealing surfaces.

### BACKGROUND OF THE INVENTION

Corresponding methods for producing a gathered or gatherable material with corresponding tools are known from the prior art. The tool can be, for example, a sonotrode of an ultrasonic processing device or a roller that is thermally heated. The following description is based on an ultrasonic machining process. However, the method according to the invention can also be used to operate other tools without ultrasound.

In order to process a material with ultrasound, the tool, e.g. the sonotrode of the device, is set into ultrasonic vibration. This results in localized heating of the material located in the gap between the sealing surface and the counter-sealing surface. The localized heating in turn causes the different layers of material arranged in the gap to fuse together and thus form a firm bond that cannot be separated non-destructively.

In contrast to ultrasonic processing, where the heating occurs in a localized area, a roller that is heated thermally heats the material over a large area. However, the other operating principle is similar.

In order to produce a gatherable or gathered material, it is necessary to combine materials with different elasticity with each other in the processing operation. For this purpose, for example, a thread is placed between the two material web sections that is significantly more elastic than the material web sections themselves. During the machining process, the thread is tensioned and clamped between the material web sections in at least two spatial directions by fusing them together.

For this purpose, in processes known from the prior art, only the area of the material web sections in which no thread is arranged is welded together. In other words, only the material web sections are welded together, but not the thread with the material web sections. In the tensioned state, the thread can still move freely within the cavity bounded by the material web sections.

If the processed material is now removed from the device and the thread is released, the frictional forces between the thread and the material web sections cause the material to gather, for example. Alternatively, the material can also be gathered in a subsequent work step and the thread shortened accordingly. Typical applications for such gathered or gatherable materials can be found, for example, in hygiene articles such as diapers.

The gathering properties of a material are therefore largely determined by the friction properties between the thread and the material web sections, which in turn are related to the properties of the materials used per se, but also to the tension of the thread during the processing operation.

The processes must therefore be individually adapted to the materials used in order to achieve sufficient gathering of the material. This requires a great deal of time. Nevertheless, the materials can still unfold if the friction is not sufficient. The known methods can therefore only be used with certain elastic threads.

### SUMMARY OF THE INVENTION

The present invention is therefore based on the problem of providing a method for producing a gathered or gatherable material, with which a material with reproducible and reliable gathering is produced. With regard to the gathered material, the present invention is based on the problem of providing a gatherable or gathered material which can be produced easily and which exhibits reliable gathering.

According to the invention, this problem is solved by a method for producing a gathered or gatherable material of the type mentioned at the beginning, the method comprising the following steps:
a) providing at least two material web sections and at least one thread, which is arranged between the material web sections,
b) providing a processing device having a tool with a sealing surface and a counter-tool with a counter-sealing surface, the tool and the counter-tool being arranged relative to one another in such a way that a gap is formed between the sealing surface and the counter-sealing surface, the tool and/or the counter-tool having at least one groove for at least partially receiving the at least one thread,
c) passing the at least two material web sections and the at least one thread through the gap between the sealing surface of the tool and the counter-sealing surface in the feed direction, the thread being guided through the at least one groove of the sealing surface or of the counter-tool, so that the two material web sections are welded together at at least two connecting surfaces, the thread being arranged between the two connecting surfaces such that there is a positive connection between the thread and the material web sections in a first and a second direction, the two directions being aligned perpendicular to one another, a cross-section of the thread relative to a cross-section of the groove being selected during the processing such that during step c. not only the two material web sections are welded together, but also an outer section of the thread is welded to the material web sections, so that the outer section of the thread is firmly connected to the two material web sections.

The thread can be firmly connected to the material web sections either by material bonding or by embedding at least one section of the thread or at least one filament of the thread in the material of the material web sections. For example, the material of the material web sections can be melted during welding, a thread section or a thread filament can be incorporated in the melt and the melt can then be solidified again. The firm connection is therefore inseparable or can only be released if the material from which the material webs are made is reheated. Relative movement between the filament and the material webs in the direction of the filament is prevented by the firm connection.

For this purpose, the tool is subjected to an ultrasonic vibration or thermally heated during step c., for example, and pressed onto the material to be processed so that the material web sections and the outer section of the thread fuse together, i.e. are firmly bonded. This connection can be a material bond. For the purposes of the present application, material bonding means that the materials to be processed form a bond with each other at least in places at the molecular level, which cannot be released non-destructively.

It is essential that the thread in the firmly connected areas can only be moved together with the material web sections. If the thread is tensioned during processing in c. and is released after processing, the material web sections gather together as the thread contracts again.

In a preferred embodiment, the connection is made in such a way that, in a sectional view perpendicular to the thread direction, the thread is only connected to the material web sections in sections in the circumferential direction, with the connection preferably being made in such a way that, in a sectional view perpendicular to the thread direction, the thread is connected to the material web sections at two separate sections in the circumferential direction. The thread is therefore not continuously connected to the material web sections in the circumferential direction. It is also not necessary to connect the thread continuously to the material web sections in the longitudinal direction. Instead, in a preferred embodiment, the connection is also only made in sections in the longitudinal direction, i.e. in the direction of the thread.

When producing a gathered material using the method according to the invention, the thread is therefore no longer freely movable between the material web sections, but is also fixed to the material web sections in the feed direction. In this way, the position of the thread in the material web sections can be determined particularly precisely. Due to the different elasticity of the materials joined together and the tensioning of the thread during welding, the material is nevertheless gathered. The products created using this process have a high level of accuracy and reproducible quality, as the threads are reliably arranged in a fixed position between the material web sections.

In one embodiment, the grooves for holding the at least one thread and the cross-section of the thread are selected such that the cross-section of the thread adapts to the shape of the grooves during step c. . . . The thread therefore does not retain its normally circular cross-section, but adapts to the shape of the grooves.

In a further embodiment, the cross-section of the thread is selected such that the cross-section of the thread in a tensioned state of the thread is larger than a depth of the groove. In other words, the thread protrudes beyond the groove and is thus processed by the tool at least in an outer section in such a way that it fuses with the material web sections.

In a further embodiment, the tool is excited in step c. with an ultrasonic vibration. The tool is therefore a sonotrode that transmits an ultrasonic vibration to the material.

In a further embodiment, the tool is heated during step c. so that the material web sections melt in the areas that come into contact with the sealing surface of the tool and are joined together at these connecting surfaces.

In a further embodiment, the groove divides the sealing surface and the counter-sealing surface, respectively, into partial sealing surfaces, the groove having a groove base and two side walls adjoining the groove base, and the side walls connecting the groove base to the partial sealing surfaces, at least one of the partial sealing surfaces having, in a sectional view perpendicular to the feed direction, at least one flank sloping down towards the groove base, the flank preferably being convexly curved with a radius R_{F} in a sectional view perpendicular to the feed direction.

In other words, the transition between the partial sealing surfaces and the groove is preferably designed in such a way that there are no longer any sharp edges that could make it difficult to change the threads between the grooves or damage the threads.

The design of the partial sealing surfaces between the grooves according to the invention reduces damage to the thread and thus the probability of thread breakage on the one hand, and makes it easier to position threads in the individual grooves on the other. As the threads slide more easily into the grooves via the flank, it is less common for a thread to be positioned in the area of the partial sealing surfaces and the risk of unintentional welding of the threads is reduced. In fact, the reliability of the thread being welded to the material web sections at the desired position is increased.

In a preferred embodiment, the radius R_{F} is between 0.01 mm and 0.5 mm and particularly preferably between 0.05 mm and 0.2 mm.

The design of the flank as a convex curvature offers the advantage that there are no longer any edges between the partial sealing surface and the side wall of the groove, but the partial sealing surface merges into the side wall of the groove in a uniform curvature. The thread cannot tilt at a corner and thus be sealed in the wrong position.

In a further embodiment, the groove base is concavely curved, with the concavely curved groove base preferably directly adjoining the convexly curved flank of the partial sealing surface.

For the purposes of the present invention, the term convex means that a straight line between two arbitrarily selectable points of the convexly curved surface extends completely within the tool or counter-tool body. The term concave, on the other hand, means that a straight line between two arbitrarily selectable points of the surface extends completely outside the body.

The concave curvature of the groove base and in particular the direct transition of the concave curved groove base into the convex curved flank offers the advantage that the groove of the device according to the invention no longer has any corners or edges in which fibers of the material to be processed could accumulate or on which the thread could get stuck. This prevents clogging of the grooves, which would result in inadequate thread guidance, or thread welding in unwanted places.

In a further embodiment, the groove base is concavely curved in a sectional view perpendicular to the feed direction with a radius R_{G}, wherein preferably 5 R_{F}>R_{G}>0.5 R_{F} and particularly preferably 2.5R_{F}>R_{G}>0.8R_{F}. The radius of curvature R_{G} of the groove base is selected in relation to the radius of curvature R_{F} of the flank in such a way that the partial sealing surfaces have a sufficient width, i.e. are not tapered. This reduces wear on the partial sealing surfaces.

In a further embodiment, the at least one groove is arranged on the sealing surface of the tool. This offers the advantage that the counter tool can be equipped with a smooth counter-sealing surface, which means that the counter tool can be manufactured very cost-effectively.

In a further embodiment, the counter tool is cylindrical and rotatable about a first cylindrical axis, the counter tool having a lateral surface on which the counter sealing surface is arranged, the first cylindrical axis being oriented in such a way that the feed direction is tangential to the lateral surface. The choice of a cylindrical counter tool offers the advantage that the material to be processed can be moved over the counter tool in the feed direction by rolling over the counter tool. Sharp edges that could damage the material to be processed are thus avoided. In addition, the reduced abrasion produces fewer fibers that could be deposited on the tool parts and thus lead to a reduced service life of the device.

In a further embodiment of the present invention, the sealing surface of the tool is essentially flat in the feed direction. As a result, the force applied by the tool to the material system to be processed can be evenly distributed.

Depending on the material feed, however, it can be advantageous if the sealing surface has a rounded edge in a sectional view parallel to the feed direction, at least in an introduction direction from which the material is introduced, so that the material to be processed, which runs into the gap at the tool, is also not damaged by sharp edges of the tool. This minimizes the tearing of threads, which also results in fewer fiber deposits in the grooves.

In a further embodiment, the groove extends completely over an extension of the sealing surface and/or the counter-sealing surface in the feed direction. This is necessary so that the thread is guided in a particularly stable manner over the entire processing width of the device according to the invention and so that no unintentional processing of the thread by the processing device occurs at any point.

In a further embodiment, the tool is cylindrical, wherein the tool is rotatable about a second cylindrical axis and has a lateral surface on which the sealing surface is arranged, wherein preferably the at least one groove runs in a circle around the lateral surface of the tool. Just as with the cylindrical counter tool, a cylindrical tool offers the advantage that material damage is prevented and the material can roll over the tool.

In a further embodiment, the sealing surface and/or the counter-sealing surface has a plurality of grooves oriented in the feed direction for at least partially receiving the at least one thread, the grooves preferably having a spacing of between 0.1 mm and 10 mm and particularly preferably between 0.4 and 2.5 mm. The distance between two grooves is defined from the center of one groove to the center of the immediately adjacent groove.

Several grooves in the sealing surface of the tool or the counter-sealing surfaces allow several threads to be arranged simultaneously between the material webs, which improves the gathering properties of a material. In addition, a uniform gathering over the desired material width can be produced in this way. The strength of the gathering is determined, among other things, by the number of threads that exert a force on the material web sections due to their restoring force in the un-tensioned state.

In a further embodiment, a plurality of grooves are combined in groove packets, with the grooves of a groove packet preferably having a spacing between 0.1 and 1 mm, with the groove packets preferably having a spacing between 5 and 300 mm from one another. Analogous to the distance between the grooves, the distance between the groove packets is defined from the center of one groove packet to the center of the adjacent groove packet.

In a further embodiment, the at least one groove has a depth of between 0.05 mm and 0.8 mm. At this depth, threads can be guided with sufficient precision and, at the same time, the grooves are comparatively easy to produce from a manufacturing point of view. Deeper grooves could lead to instability of the tool, especially if the distance between the grooves is small, so that only narrow partial sealing surfaces remain, but these are arranged comparatively far away from the groove base. If both the groove base and the flank are curved, the depth of the groove is preferably determined by the sum of the radius of curvature R_{F} of the flank and the radius of curvature R_{G} of the groove base.

In a further embodiment, the partial sealing surfaces are convexly curved in a sectional view perpendicular to the feed direction with a radius of curvature RT, wherein preferably the partial sealing surfaces merge into the flanks and preferably R_{F}=RT. In other words, the partial sealing surface provides a convex curvature which preferably has a radius of curvature which corresponds to the radius of curvature of the flanks. The partial sealing surfaces therefore no longer have a flat surface, but are curved at every point. In this way, only a line sealing of the material webs takes place in the feed direction. This is particularly effective in preventing unwanted processing of the thread.

With regard to the gatherable or gathered material, the problem mentioned at the beginning is solved by a gatherable or gathered material consisting of at least two material web sections and at least one thread which is arranged between the material web sections, the two material web sections being welded together at at least two connecting surfaces, the thread being arranged between the two connecting surfaces in such a way that there is a positive connection between the thread and the material web sections in a first and a second direction, the two directions being aligned perpendicular to one another, characterized in that the thread is firmly connected to the two material web sections.

It is sufficient if only filaments of the thread are firmly connected to the material web sections.

Preferably, the gatherable or gathered material is produced using the process described.

### BRIEF DESCRIPTION OF THE DRAWINGS

Further advantages, features and possible applications will become clear from the following description of an embodiment and the associated figures.

FIG. 1 shows a sectional view of a section of a tool which is operated using the method according to the invention.

FIG. 2a schematically shows the arrangement of the thread between the material webs after welding using methods known from the prior art.

FIG. 2b schematically shows the arrangement of the thread according to one embodiment of the method according to the invention.

### DETAILED DESCRIPTION OF THE INVENTION

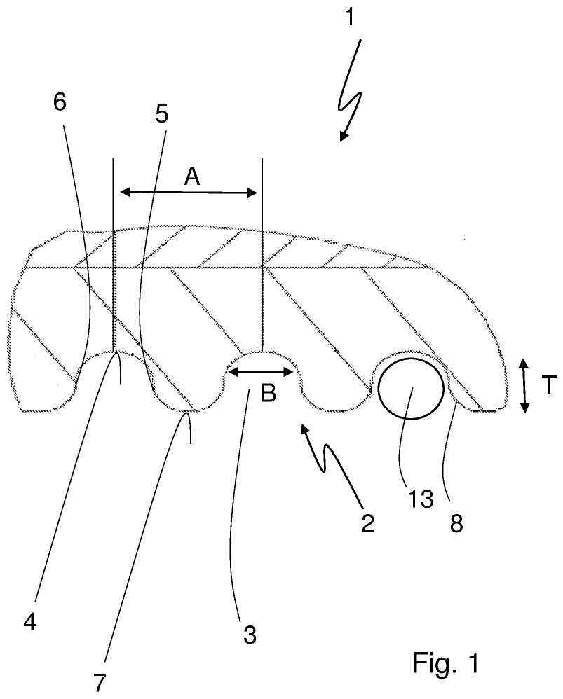

According to the embodiment of the device according to the invention shown in FIG. 1, the grooves 3 for receiving the thread 13 are arranged in the sonotrode sealing surface 2 of a sonotrode 1. The grooves 3 run in the feed direction, which extends into the sheet plane in the sectional view shown in FIG. 1. In addition, the grooves 3 divide the sonotrode sealing surface 2 into several partial sealing surfaces 7.

The grooves 3 have a groove base 4 and two side walls 5, 6 adjoining the groove base, whereby the side walls 5, 6 connect the groove base 4 with the partial sealing surfaces 7.

In the sectional view shown in FIG. 1, the partial sealing surfaces 7 each have two flanks 8 sloping down towards the groove base 4 perpendicular to the feed direction.

The sloping flanks 8 are convexly curved with a radius R_{F}, so that the entire partial sealing surface 7 is convexly curved with a radius RT=R_{F}. Furthermore, the groove base 4 is also concavely curved with a radius R_{G} and directly adjoins the convexly curved flanks of the partial sealing surfaces 7. The radius RT of the curved partial sealing surface 7 corresponds to the radius R_{G} of the concavely curved groove base 4 and is 0.1 mm.

The grooves also have a width B of 0.22 mm, whereby the width is measured between the two transition points of the concavely curved groove base 4 and the convexly curved flank 8.

In addition, the grooves 3 have a depth T of 0.21 mm. The depth T of the grooves 3 is defined between the deepest point of the groove base 4 and the highest point of the partial sealing surface 7. The distance A between two grooves is 0.42 mm, whereby the distance A is measured at the deepest point of the groove base 4.

To produce a gatherable or gathered material 10, two material webs 11, 12 are arranged between the sonotrode sealing surface 2 of the sonotrode 1 and the counter-sealing surface of a counter tool (not shown), between which a thread 13 is arranged. The thread 13 is guided in such a way that it runs inside the grooves 3. Ultrasonic processing of the material web sections 11, 12 only occurs in the area of the partial sealing surfaces 7.

In methods known from the prior art, a diameter of the thread 13 in a state in which the thread is tensioned is selected so that it is smaller than a depth of the groove 3. This means that the thread 13 is not processed by the sonotrode 1.

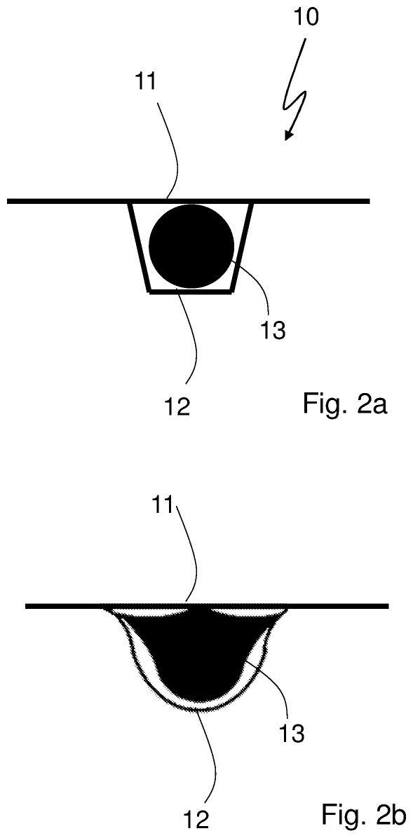

The result of such a process with devices known from the prior art, i.e. with devices whose grooves have sharp edges, is shown in FIG. 2a. The thread 13 is clamped in a cavity between the material web sections 11, 12 of the material 10, but can move in the feed direction relative to the material web sections 11, 12. The lower material web section 12 has the shape of the grooves, which can be arranged on the sonotrode or the counter tool or both.

According to the method according to the invention, the cross-section of the thread 13 is selected so that it is larger than a depth of the groove 3 even in the tensioned state. The result of the method according to the invention is shown in FIG. 2b. By selecting the larger cross-section of the thread 13, the thread 13 is welded in the area of the partial sealing surface 7. As a result, the thread 13 is also attached to the material web sections 11, 12 and can no longer be moved in the feed direction. The originally circular cross-section of the thread 13 has also adapted to the shape of the groove 3.

Moreover, in a preferred embodiment, the distance between the at least two connecting surfaces during welding can be selected such that this distance either i) corresponds to the diameter of the thread 13 in a relaxed state or ii) is larger than the diameter of the thread 13 in a relaxed state. Due to the firm connection according to the invention, it is therefore no longer necessary to select a large diameter of the thread 13 in the relaxed state in order to realize friction between material web sections 11 and the thread 13.

In this way, a gathered or gatherable material can be produced whose threads are always arranged in the same position.

### LIST OF REFERENCE SIGNS

1 Sonotrode
2 Sonotrode sealing surface
3 Groove
4 Groove base
5, 6 Side walls
7 Partial sealing area
8 Flank
10 Gathered or gatherable material
11, 12 Material web sections
13 Thread

What is claimed is:

1. A method for producing a gathered or gatherable material, the method comprising the following steps:
providing at least two material web sections and at least one thread, which is arranged between the material web sections,
providing a processing device having a tool with a sealing surface and a counter-tool with a counter-sealing surface, the tool and the counter-tool being arranged relative to one another in such a way that a gap is formed between the sealing surface and the counter-sealing surface, the tool and/or the counter-tool having at least one groove for at least partially receiving the at least one thread,
passing the at least two material web sections and the at least one thread through the gap between the sealing surface of the tool and the counter-sealing surface in a feed direction, the thread being guided through the at least one groove of the sealing surface and/or of the counter-tool,
so that the two material web sections are welded together at at least two connecting surfaces, the thread being arranged between the two connecting surfaces in such a way that there is a positive connection between the thread and the material web sections in a first and a second direction, the two directions being aligned perpendicular to one another, characterized in that a cross section of the thread relative to a cross section of the groove is selected during processing in such a way that during step c. not only the two material web sections are welded together, but also an outer section of the thread is welded to the material web sections, so that the outer section of the thread is firmly connected to the two material web sections.

2. The method according to claim 1, wherein the grooves for receiving the at least one thread and the cross-section of the thread are selected such that the cross-section of the thread adapts to the groove shape during step c.

3. The method according to claim 1, wherein the cross-section of the thread is selected such that the cross-section of the thread in a tensioned state of the thread is larger than a depth of the groove.

4. The method according to claim 1, wherein the tool is excited with an ultrasonic vibration in step c., so that the two material web sections are processed with ultrasound at the at least two connecting surfaces.

5. The method according to claim 1, wherein the tool is heated in step c. so that the two material web sections fuse together at the at least two connecting surfaces.

6. The method according to claim 1, wherein the groove is oriented in the feed direction, wherein the groove divides the sealing surface and the counter-sealing surface, respectively, into partial sealing surfaces, wherein the groove has a groove base and two side walls adjoining the groove base, and wherein the side walls connect the groove base to the partial sealing surfaces, wherein at least one of the partial sealing surfaces having, in a sectional view perpendicular to the feed direction, at least one flank sloping down towards the groove base, the flank preferably being convexly curved with a radius R_{F} in a sectional view perpendicular to the feed direction.

7. The method according to claim 6, wherein the groove base is concavely curved, the concavely curved groove base preferably directly adjoining the preferably convexly curved flank of the partial sealing surface.

8. The method according to claim 1, wherein the at least one groove is arranged on the sealing surface of the tool.

9. The method according to claim 1, wherein the sealing surface and/or the counter-sealing surface comprise a plurality of grooves oriented in the feed direction for at least partially receiving the at least one thread.

10. The method according to claim 1, wherein the at least one groove has a depth between 0.05 mm and 0.8 mm.

11. The method according to claim 1, wherein the distance between the at least two connecting surfaces during welding either i) corresponds to the diameter of the thread in a relaxed state or ii) is larger than the diameter of the thread in a relaxed state.

12. The method according to claim 1, wherein the firm connection is made in such a way that, in a sectional view perpendicular to the thread direction, the thread is firmly connected to the material web sections only in sections in the circumferential direction, the firm connection preferably being made in such a way that, in a sectional view perpendicular to the thread direction, the thread is firmly connected to the material web sections in the circumferential direction at two sections separated from one another.

13. A gatherable or gathered material consisting of at least two material web sections and at least one thread which is arranged between the material web sections, the two material web sections being welded to one another at at least two connecting surfaces, the thread being arranged between the two connecting surfaces in such a way that there is a positive connection between the thread and the material web sections in a first and a second direction, the two directions being aligned perpendicular to one another, wherein the thread is firmly connected to the two material web sections.

14. The gatherable or gathered material according to claim 13, wherein it has been produced by a method, the method comprising the following steps:
providing at least two material web sections and at least one thread, which is arranged between the material web sections,
providing a processing device having a tool with a sealing surface and a counter-tool with a counter-sealing surface, the tool and the counter-tool being arranged relative to one another in such a way that a gap is formed between the sealing surface and the counter-sealing surface, the tool and/or the counter-tool having at least one groove for at least partially receiving the at least one thread,
passing the at least two material web sections and the at least one thread through the gap between the sealing surface of the tool and the counter-sealing surface in a feed direction, the thread being guided through the at least one groove of the sealing surface and/or of the counter-tool,
so that the two material web sections are welded together at at least two connecting surfaces, the thread being arranged between the two connecting surfaces in such a way that there is a positive connection between the thread and the material web sections in a first and a second direction, the two directions being aligned perpendicular to one another, characterized in that a cross section of the thread relative to a cross section of the groove is selected during processing in such a way that during step c. not only the two material web sections are welded together, but also an outer section of the thread is welded to the material web sections so that the outer section of the thread is firmly connected to the two material web sections.

15. The method according to claim 1, wherein the cross-section of the thread is selected such that the cross-section of the thread in a tensioned state of the thread is larger than a depth of the groove, and wherein the tool is excited with an ultrasonic vibration in step c., so that the two material web sections are processed with ultrasound at the at least two connecting surfaces.

16. The method according to claim 15, wherein the groove is oriented in the feed direction, wherein the groove divides the sealing surface and the counter-sealing surface, respectively, into partial sealing surfaces, wherein the groove has a groove base and two side walls adjoining the groove base, and wherein the side walls connect the groove base to the partial sealing surfaces, wherein at least one of the partial sealing surfaces having, in a sectional view perpendicular to the feed direction, at least one flank sloping down towards the groove base, the flank preferably being convexly curved with a radius R_{F} in a sectional view perpendicular to the feed direction.

17. The method according to claim 16, wherein the groove base is concavely curved, the concavely curved groove base preferably directly adjoining the preferably convexly curved flank of the partial sealing surface, and wherein the at least one groove is arranged on the sealing surface of the tool.

18. The method according to claim 17, wherein the sealing surface and/or the counter-sealing surface comprise a plurality of grooves oriented in the feed direction for at least partially receiving the at least one thread, and wherein the at least one groove has a depth between 0.05 mm and 0.8 mm.

19. The method according to claim 15, wherein the distance between the at least two connecting surfaces during welding either i) corresponds to the diameter of the thread in a relaxed state or ii) is larger than the diameter of the thread in a relaxed state, and wherein the firm connection is made in such a way that, in a sectional view perpendicular to the thread direction, the thread is firmly connected to the material web sections only in sections in the circumferential direction, the firm connection preferably being made in such a way that, in a sectional view perpendicular to the thread direction, the thread is firmly connected to the material web sections in the circumferential direction at two sections separated from one another.

20. A gatherable or gathered material consisting of at least two material web sections and at least one thread which is arranged between the material web sections, the two material web sections being welded to one another at at least two connecting surfaces, the thread being arranged between the two connecting surfaces in such a way that there is a positive connection between the thread and the material web sections in a first and a second direction, the two directions being aligned perpendicular to one another, wherein the thread is firmly connected to the two material web sections, produced by the method according to claim 19.
